(19) United States
(12) Patent Application Publication
(10) Pub. No.: US 2023/0181196 A1
(43) Pub. Date: Jun. 15, 2023

(54) REPOSITIONABLE OVER THE SCOPE CLIP

(71) Applicants: BOSTON SCIENTIFIC MEDICAL DEVICE LIMITED, Galway (IE); BOSTON SCIENTIFIC SCIMED, INC., Maple Grove, MN (US)

(72) Inventors: Sharath Kumar G, Kanakapura (IN); Deepak Kumar SHARMA, Muzaffarnagar (IN); Paul SMITH, Smithfield, RI (US); Arun ADHIKARATH BALAN, Aluva (IN); Rajivkumar SINGH, Thane (IN)

(21) Appl. No.: 18/065,389

(22) Filed: Dec. 13, 2022

Related U.S. Application Data
(60) Provisional application No. 63/265,382, filed on Dec. 14, 2021.

(51) Int. Cl. A61B 17/128; A61B 17/122

(52) U.S. Cl. CPC A61B 17/1285; A61B 17/122

(57) ABSTRACT

A system includes an adapter mounted over a distal end of an insertion device and an extending member. A clip mounted over the adapter includes jaws movable to separate from one another and, when the clip is moved off the adapter, to draw toward one another. The first member is releasably coupled to the clip, movably connected to the adapter and includes a clasp releasably coupling the clip to the first member via an engaging portion of one jaw to permit movement of the adapter relative to the clip while the first member remains coupled to the clip, enhancing visualization of the clip. The first member is operable to retract the clip proximally over the adapter so that the clip is forced open as the clip is retracted over the adapter freeing the clip from tissue on which it had been clipped.

The present disclosure claims priority to U.S. Provisional Patent Application Ser. No. 63/265,382 filed Dec. 14, 2021; the disclosure of which is incorporated herewith by reference.

### FIELD

The present disclosure relates to endoscopic devices and, in particular, relates to endoscopic clipping devices for treating tissue along the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of the procedure. Such procedures may include, for example, the removal of large lesions, tunneling under the mucosal layer of the GI tract to treat issues below the mucosa, full thickness removal of tissue, treatment of issues on other organs by passing outside of the GI tract, and endoscopic treatment/repair of post-surgical issues (e.g., post-surgical leaks, breakdown of surgical staple lines, and anastomotic leaks).

Currently, tissue openings (e.g., surgical openings, lesions, etc.) may be treated via endoscopic closure devices including through-the scope clips or over-the-scope clips. Over-the-scope clips may be particularly useful for achieving closure of larger tissue openings. These endoscopic closure devices can save costs for the hospital and may provide benefits for the patient. In some cases, current endoscopic closure devices may be difficult to use, time consuming to position, or insufficient for certain perforations, conditions and anatomies. For example, current over-the-scope clips generally require launching of the clip from a position in which the clip itself is not visible to the operator. That is, prior to clipping the operator may view the target tissue to be clipped and, based on this visualization of the target tissue may determine that the distal end of the device and the clip are in a desired position relative to the target tissue. Based on the observation of the target tissue, the operator then deploys the clip without being able to see the clip itself until it has been deployed. Once deployed, such current over-the scope clips are generally incapable of being repositioned.

### SUMMARY

The present disclosure relates to a clipping system for treating tissue. The system includes an adapter including a proximal portion configured to be mounted over a distal end of an insertion device and a distal portion extending distally from the proximal portion; a clip; and a first extending member. The clip is configured to be mounted over the adapter, the clip including first and second jaws connected to one another such that the first and second jaws are movable between an insertion configuration, in which the first and second jaws extend about opposing portions of the distal portion of the adapter and are separated from one another to receive tissue therebetween, and an initial deployed configuration, in which the clip is moved distally off of the adapter so that the first and second jaws are drawn toward one another to grip tissue therebetween. The first and second jaws are biased toward the initial deployed configuration. The first extending member are configured to be releasably coupled to the clip and movably connected to the adapter. The first extending member includes a clasp at a distal end thereof. The clasp includes a first arm and a second arm pivotally coupled to one another so that distal ends thereof are movable toward and away from one another between an engaging configuration and a disengaging configuration, respectively, the clasp of the first extending member engaging an engaging portion of the first jaw to permit movement of the adapter relative to the clip while the first extending member remains coupled to the clip to place the system in a review configuration in which the clip is physically separated from the adapter to enhance visual observation of the clip. The first extending member is operable to retract the clip proximally over the adapter so that the clip is forced open as the clip is retracted over the adapter freeing the clip from tissue on which it had been clipped.

In an embodiment, the first jaw includes an opening extending therethrough, the engaging portion extending between the opening of the first jaw and an exterior edge of the first jaw.

In an embodiment, the first arm of the clasp is substantially hook-shaped, the first arm extending along a curve from a proximal end toward a distal hook tip, the first arm configured to be hooked through the opening of the first jaw so that the engaging portion of the first jaw is received within the curve of the first arm.

In an embodiment, proximal ends of the first and second arms of the clasp are pivotally coupled to one another so that the first and second arms pivot between the engaging configuration, in which the distal hook tip and a distal end of the second arm contact one another to enclose the engaging portion of the first jaw therewithin, and the disengaging configuration, in which the distal hook tip and the distal end of the second arm are separated from one another to release the engaging portion of the first jaw therefrom.

In an embodiment, the insertion device further includes a first control wire slidably received within the first extending member, a distal end of the first control wire connected to the first and second arms of the clasp at the distal end of the first extending member.

In an embodiment, the first control wire is connected to the clasp of the first extending member via a four-bar mechanism so that a longitudinal movement of the first control wire relative to the first extending member moves the clasp of the first extending member between the engaging and disengaging configurations.

In an embodiment, the proximal portion of the adapter includes a first hole extending longitudinally through a wall thereof to slidably receive the first extending member therein such that the clasp at the distal end of the first extending member extends distally of the first hole to engage the clip.

In an embodiment, the system further includes a second extending member configured to be releasably coupled to the clip and movably connected to the adapter, the second extending member including a clasp at a distal end thereof, the clasp of the second extending member including a first arm and a second arm pivotally coupled to one another so that distal ends thereof are movable toward and away from one another between an engaging configuration and a disengaging configuration, respectively, the clasp of the second extending member engaging an engaging portion of the second jaw to permit movement of the clip relative to the adapter between the insertion configuration, the initial deployed configuration and the review configuration.

In an embodiment, the insertion device further includes a second control wire slidably received within the second extending member, a distal end of the second control wire connected to the first and second arms of the clasp at the distal end of the second extending member.

In addition, the present disclosure relates to a clipping system for treating tissue. The system includes an endoscope extending longitudinally from a proximal end to a distal end; an adapter; a clip; and first and second extending members. The adapter includes a proximal portion and a distal portion. The proximal portion is configured to be mounted over the distal end of the endoscope so that a channel of the adapter is aligned with a channel of the endoscope. The clip is configured to be mounted over the distal portion of the adapter. The clip includes first and second jaws connected to one another such that the first and second jaws are movable between an insertion configuration, in which the first and second jaws extend about opposing portions of the distal portion of the adapter and are separated from one another to receive tissue therebetween, and an initial deployed configuration, in which the clip is moved distally off of the adapter so that the first and second jaws are drawn toward one another to grip tissue therebetween. The first and second jaws is biased toward the initial deployed configuration, each of the first and second jaws including an opening extending therethrough to define an engaging portion. The first and second extending members are configured to be releasably coupled engaging portions of the first and second jaws of the clip and movably connected to the adapter. Each of the first and second extending member includes a clasp at a distal end thereof. Each clasp includes a first arm and a second arm pivotally coupled to one another so that distal ends thereof are movable toward and away from one another between an engaging configuration and a disengaging configuration, respectively, so that when the first and second extending members engage the clip in the engaging configuration, longitudinal movement of the first and second extending members relative to the endoscope moves the clip between the insertion configuration, the initial deployed configuration and a review configuration in which the clip is physically spaced from the adapter to enhance visual observation of the clip.

In an embodiment, the first arm of each clasp is substantially hook-shaped, the first arm extending along a curve from a proximal end toward a distal hook tip, the first arm configured to be hooked through a corresponding opening of one of the first and second jaws so that the engaging portion defined via the opening and an exterior edge of the corresponding one of the first and second jaws is received within the curve of the first arm.

In an embodiment, proximal ends of the first and second arms of each clasp are pivotally coupled to one another so that the first and second arms pivot between the engaging configuration, in which the distal hook tip and a distal end of the second arm contact one another to enclose the engaging portion of the corresponding one of the first and second jaws therewithin, and the disengaging configuration, in which the distal hook tip and the distal end of the second arm are separated from one another to release the engaging portion of the corresponding one of the first and second jaws therefrom.

In an embodiment, the endoscope further includes first and second control wires, the first and second control wires slidably received within the first and second extending members, respectively, so that distal ends of each of the first and second control wires are connected to the first and second arms of the clasp of each of the first and second extending members.

In an embodiment, the system further includes a user interface including a handle member, a spool slidably mounted over the handle member, and a button slidably coupled to the spool, the spool connected to proximal ends of the first and second extending members so that sliding a spool longitudinally relative to the handle member correspondingly moves the first and second extending members relative to the endoscope, the button connected to proximal ends of the first and second control wires so that siding the button longitudinally relative to the spool correspondingly moves the first and second control wires relative to the first and second extending members.

The present disclosure also relates to a method for treating tissue. The method includes inserting a clip to a target area in a body lumen via an endoscope, the clip mounted over a distal end of the endoscope, via an adapter, in an open insertion configuration in which first and second jaws of the clip are separated from one another; drawing tissue into a channel of the adapter and between the jaws of the clip; moving the clip from the open insertion configuration toward an initial deployed configuration by releasing a tension along extending members coupled to the clip so that the jaws revert to a biased closed configuration, in which the jaws extend toward one another to grip the tissue received therebetween, the extending members releasably coupled to the clip via clasps at distal end of the extending members, each of the clasps including first and second arms pivotally coupled to one another, distal ends of the first and second arms contacting one another to enclose engaging portions of the clip therebetween in an engaging configuration;

and moving the endoscope relative to the clip, while the extending members remain coupled to the clip, to a review configuration in which visualization of the clip via the endoscope is enhanced.

In an embodiment, when it is determined that the clip requires repositioning, moving the extending members proximally relative to the endoscope until the clip is drawn proximally over the adapter toward the open insertion configuration and repositioning the clip over the target tissue.

In an embodiment, the method further includes moving the clip from the review configuration toward a final deployed configuration by moving the clasps toward a disengaging configuration, in which distal ends of the clasps are moved away from one another toward a disengaging configuration to release the engaging portions of the clip from therebetween.

In an embodiment, the clasps are moved between the engaging configuration and the disengaging configuration via control wires connected to proximal ends of the first and second arms of the clasps, the control wires slidably received within the extending members so that the control wires are longitudinally movable relative to the extending members.

In an embodiment, the first arm of each clasp is substantially hook-shaped so that the first arm extends along a curve from a proximal end toward a distal hook tip, the first arm hooked through a corresponding opening of one of the first and second jaws so that the engaging portions defined between the opening and an exterior edge of the corresponding one of the first and second jaws is received within the curve of the first arm in the engaging configuration.

### BRIEF DESCRIPTION

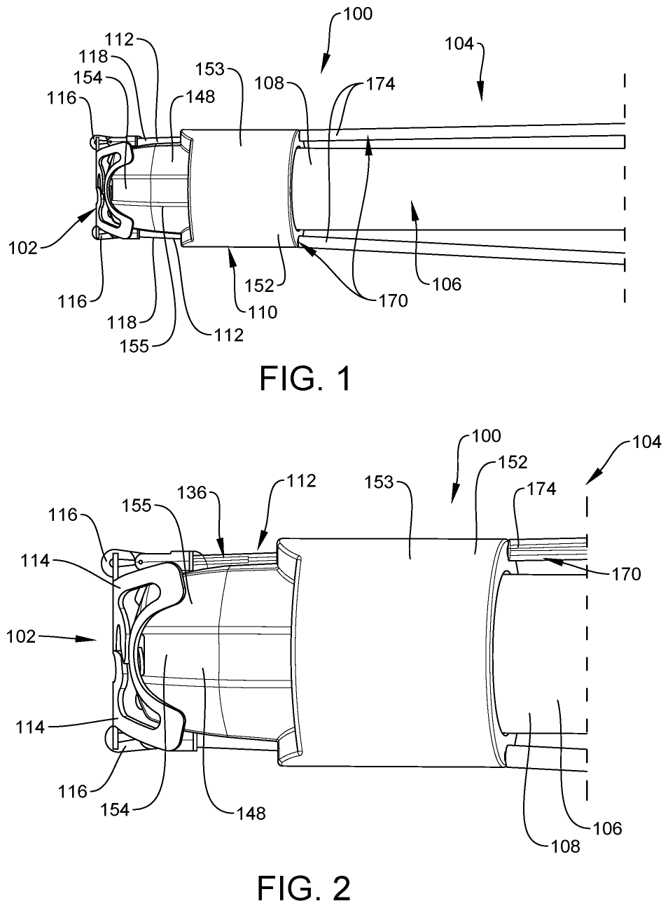

FIG. 1 shows a longitudinal side view of a distal portion of a system according to an exemplary embodiment of the present disclosure;

FIG. 2 shows a partially transparent longitudinal side view of the distal portion of the system according to FIG. 1;

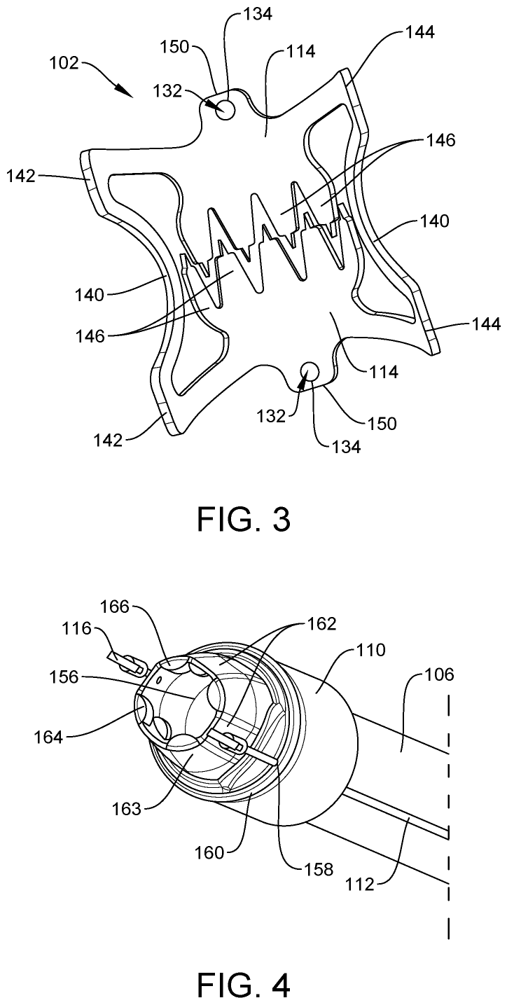

FIG. 3 shows a perspective view of a clip of the system according to FIG. 1;

FIG. 4 shows a perspective view of the distal portion of an insertion device of the system according to FIG. 1;

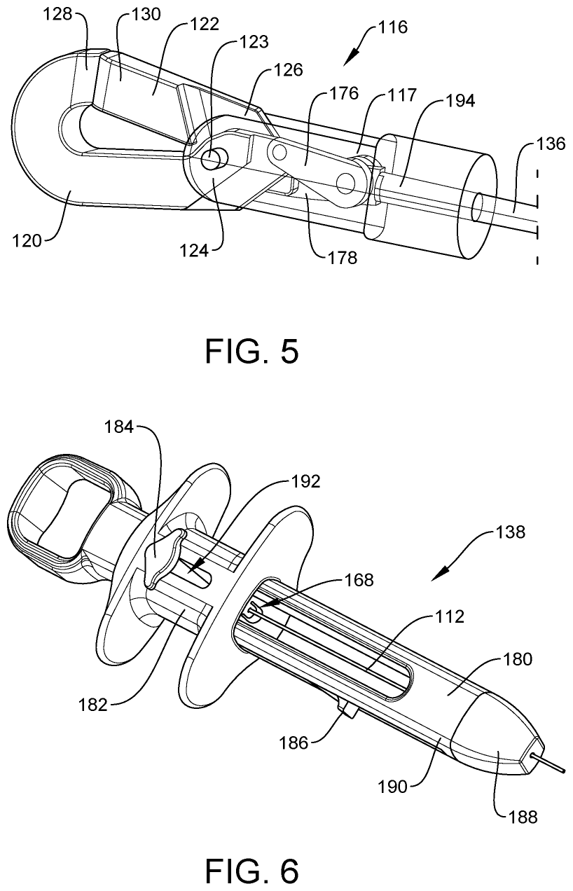

FIG. 5 shows a perspective view of a clasp of the insertion device according to the system of FIG. 1;

FIG. 6 shows a perspective view of a user interface of the system according to FIG. 1;

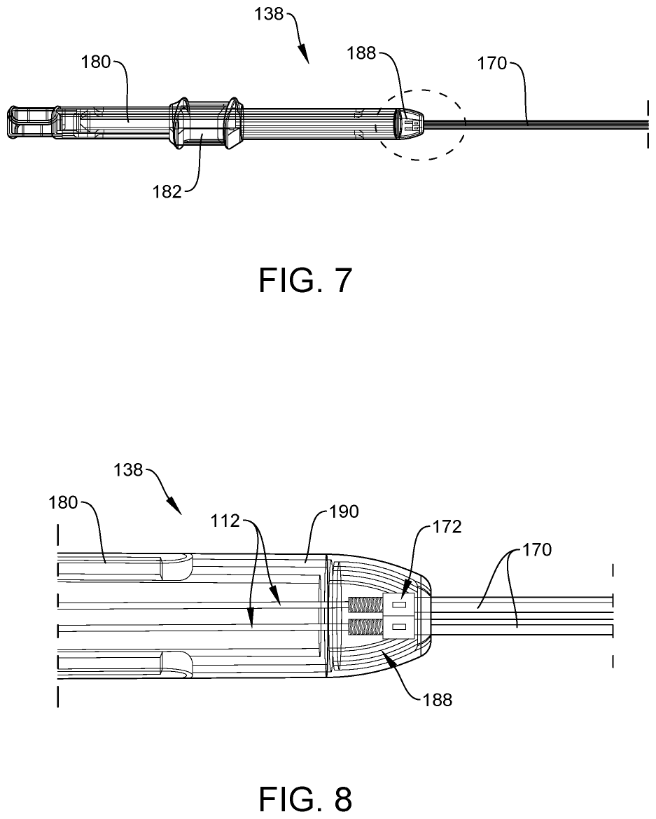

FIG. 7 shows a transparent longitudinal side view of the user interface according to the system of FIG. 1; and

FIG. 8 shows an enlarged longitudinal side view of a portion of the user interface according to the system of FIG. 1.

### DETAILED DESCRIPTION

The present disclosure may be further understood with reference to the following description and the appended drawings, wherein like elements are referred to with the same reference numerals. The present disclosure relates to a clipping system and, in particular, relates to an over-the-scope endoscopic clipping system, in which an initial placement of a clip may be viewed and adjusted prior to final deployment of the clip. Exemplary embodiments of the present disclosure comprise a clip mountable over a distal end of an endoscope via an adapter and releasably coupled to extending members so that the clip may be moved between an insertion configuration, an initial deployed configuration, and a review configuration, in which the clip can be viewed prior to being finally deployed.

According to an exemplary embodiment, each of the extending members includes a clasp at a distal end thereof, each clasp configured to engage a corresponding jaw of the clip via an opening extending therethrough. The clasp of one embodiment includes first and second arms pivotally coupled to one another such that distal ends thereof are movable toward and away from one another between an engaging configuration and a disengaging configuration, respectively.

In the engaging configuration, distal ends of the first and second arms contact one another to form an enclosure within which an engaging portion of the corresponding jaw is received. In the disengaging configuration, the first and second arms are moved away from one another so that the engaging portion of the jaw is released from the engaging portion of the corresponding jaw to separate the clip from the device. While the extending members are engaged with the clip via clasps thereof, the extending members may be moved longitudinally relative to the endoscope to move the clip between the insertion configuration, the initial deployed configuration and the review configuration.

In the insertion configuration, the clip is mounted over the adapter in a proximal position maintained in the insertion configuration with the jaws spread apart ready to receive tissue therebetween while the clip's position minimizes its occlusion of the field of view of the endoscopic vision system. The insertion configuration is configured to facilitate insertion of the endoscope to a target site adjacent to tissue to be clipped while the system allows the clip to be deployed and clipped over tissue in an initial deployed configuration. The device permits the endoscope to be withdrawn proximally away from the clip and the tissue over which it is clipped while the clip remains coupled to the device in a review configuration.

As the endoscope is withdrawn proximally while the clip remains in place clipped over the target tissue, the field of view of the vision system of the endoscope widens to show the clip and the tissue clipped thereby so that the operator can determine whether the position of the clip is desirable or in need of adjustment. If the operator determines that the clip is positioned as desired, the clip is deployed by releasing the clip from the clasps of the extending members and left in place clipped over the target tissue. If the operator determines that the position of the clip needs adjustment, the endoscope and the adapter coupled thereto are moved distally to a position adjacent to the clip.

The clip is then drawn proximally over the adapter to reopen the clip which is drawn proximally over the distal end of the adapter forcing the jaws of the clip to open against the natural bias of the clip as the clip slides proximally back over the adapter to return to the insertion configuration. After the clip has been removed from the tissue and returned to the insertion configuration, the operator can re-position the endoscope and device as desired, draw target tissue into the adapter (e.g., under suction or via a grasper inserted into the adapter through a working channel of the endoscope) and once more deploy the clip from the adapter over the target tissue in the initial deployed position. The endoscope is then withdrawn proximally once again as the clip remains coupled to the device so that the device moves again into the review configuration. The position of the clip and the clipped tissue are again observed and, this process is repeated until the clip is positioned as desired.

When the operator sees that the clip is closed over a target portion of tissue as the desired (e.g., closing an opening in the tissue), the first and second arms of each clasp are moved away from one another to the disengaging configuration to completely release the clip from the device in the final deployed configuration so that the endoscope and the adapter may be withdrawn from the body leaving the clip in place clipped over the target tissue. It will be understood by those of skill in the art that terms proximal and distal, as used herein, are intended to refer to a direction toward and away from, respectively, a user of the device.

As shown in FIGS. 1-9, a clipping system 100 for treating tissue defects and/or perforations according to an exemplary embodiment comprises a clip 102 configured to be inserted through, for example, a body lumen to a target area to clip target tissue. The clip 102 is insertable to the target area via an insertion device 104 including, for example, an endoscope 106. The clip 102 is coupled to a distal end 108 of the endoscope 106 via an adapter 110 mounted over the distal end 108 of the endoscope 106 and is movable relative to the endoscope 106 via extending members 112 which extend along the outside of the endoscope 106 to releasably couple to the clip 102.

The adapter 110 of this embodiment is a hollow member sized and shaped to be friction fit over the distal end of the endoscope 106 and is radially stiff enough so that when the clip 102 is received on the adapter 110, the adapter 110 maintains the jaws 114 of the clip 102 spread apart from one another against their natural bias without collapsing the adapter 110 (i.e., so that a distal end of the adapter is open to receive tissue to be clipped). Each of the extending members 112 includes a clasp 116 at a distal end 118 thereof configured to be releasably engaged with a portion of a corresponding jaw 114 of the clip 102 to facilitate movement of the clip 102 relative to the endoscope 106 between an insertion configuration, an initial deployed configuration, a review configuration, and a final deployed configuration.

According to an exemplary embodiment, each clasp 116 includes a first arm 120 and a second arm 122, proximal ends 124, 126 of which are pivotally connected to one another such that the first and second arms 120, 122 are movable relative to one another between an engaging configuration in which distal ends 128, 130 contact one another so that a portion of a corresponding jaw 114 of the clip 102 is clasped thereby and a disengaging configuration in which the first and second arms 120, 122 are moved away from one another to release the clip 102 from the extending members 112. While clasps 116 engage the clip 102 in the engaging configuration, longitudinal movement of the extending members 112 relative to the endoscope 106 moves the clip 102 between the insertion configuration, the initial deployed configuration and the review configuration.

In the insertion configuration, the clip 102 is mounted over the adapter 110 with jaws 114 separated from one another to receive tissue therebetween. To move the clip 102 from the insertion configuration toward the initial deployed configuration, the extending members 112 are moved distally relative to the endoscope 106, pushing the clip 102 distally off of the adapter 110 so that, as the clip 102 moves off of the adapter 110, the jaws 114 are drawn toward one another under the natural bias of the clip 102 to a closed configuration in which the jaw 114 are drawn together to grip tissue that has been drawn into the adapter 110.

Upon clipping of the tissue via the jaws 114 in the initial deployed configuration, the clip 102 may be moved toward the review configuration by moving the extending members 112 distally away from the endoscope 106 (or drawing the endoscope 106 proximally relative to the extending members 112) so that the clip 102 is distanced from the adapter 110, while remaining tethered to the insertion device 104 via the extending members 112. This widens the field of view of the endoscope vision system relative to the clip 102 and the target tissue and allows for some movement of the endoscope 106 relative to the clip 102 to enable more extensive observation of the placement and/or position of the clip 102 relative to the target tissue (e.g., from different angles, etc.).

As described below, if the user determines the position of the clip 102 is incorrect or sub-optimal, the user may move the endoscope 106 distally to a position adjacent to the clip 102. The user may then retract the clip 102 proximally over the distal end of the adapter 110 so that the adapter 110 spreads the jaws 114 apart as the clip 102 is moved into the open insertion configuration. This releases the previously clipped tissue allowing the user to reposition the endoscope 106 and the clip 102 relative to the target tissue and repeat the steps detailed above so that the placement and/or position of the clip 102 relative to a target tissue may be adjusted, as desired, prior to a final deployment of the clip 102. That is, if the operator determines in the review configuration that the clip 102 is not positioned as desired, the clip 102 may be re-opened and removed from the tissue so that the device can be re-positioned until the clip 102 has been repositioned and closed over the desired portion of tissue.

Once it has been determined that the clip 102 has been clipped over the desired tissue, the clasps 116 may be moved toward the disengaging configuration to release the clip 102 from between the first and second arms 120, 122 thereof completely freeing the clip from the extending members 112 and the insertion device 104 so that the insertion device 104 and the adapter 110 may be proximally withdrawn from the body leaving the clip 102 in the body clipped over the target tissue.

As will be understood by those of skill in the art and as will be described in further detail below, actuation of the extending members 112 to control movement of the clip 102 between the insertion configuration, the initial deployed configuration and the review configuration may be separate from movement of the clip 102 toward the final deployed configuration. In an exemplary embodiment, movement of the clip 102 from the review configuration toward the final deployed configuration may be controlled via a control wire 136 connected to the proximal ends 124, 126 of the first and second arms 120, 122 of the clasp 116 while actuation of the extending members 112 and the control wire 136 is controlled via a user interface 138 which, in one embodiment, as shown in FIGS. 6-8, is coupled to a proximal end of the insertion device 104.

As shown in FIG. 3, the clip 102 includes first and second jaws 114 connected to one another via hinges 140. In one embodiment, each of the jaws 114 extends along a curve from a first end 142 to a second end 144 with a first one of the hinges 140 connecting the first ends 142 of each of the jaws 114 to one another, while a second one of the hinges 140 connects the second ends 144 of each of the jaws 114 to one another. In one embodiment, the hinges 140 are spring biased, biasing the jaws 114 toward the initial deployed configuration in which the jaws 114 are moved toward one another, in a closed configuration to grip tissue received therebetween.

Each of the jaws 114 may include gripping features such as, for example, teeth, so that, in this initial deployed configuration, the gripping features 146 of one of the jaws 114 contact the gripping features 146 of the other jaw 114, e.g., in an interlocking pattern. In particular, in the initial deployed configuration, the jaws 114 extend toward one another so that the target tissue may be gripped between the jaws 114 via the gripping features 146. However, when the clip 102 is mounted over the adapter 110 in the insertion configuration, the jaws 114 are spread apart across opposing portions of the adapter 110 so that an exterior surface 148 of the adapter 110 maintains the clip 102 open with the jaws 114 separated from one another despite this spring bias. Thus, when the clip 102 is mounted over the adapter 110 in the insertion configuration, target tissue may be drawn into the adapter 110 between the jaws 114.

When the clip 102 is moved distally off of the adapter 110, the clip 102 closes under the natural bias of the hinges 140. It will be understood by those of skill in the art that the hinges 140 and/or jaws 114 of the clips 102 may be formed of any of a variety of materials so long as the hinges 140 bias the jaws 114 toward the initial deployed configuration, as described above, and so that the bias is sufficiently strong to maintain the clip 102 in clipped position over target tissue after the clip has been finally deployed. In one example, portions of the clip 102 (e.g., the hinges 140) are formed of a shape memory alloy such as, for example, Nitinol to provide and/or add to the bias toward the closed configuration.

According to an exemplary embodiment, as described above, each of the jaws 114 includes an opening 132 extending therethrough. Each of the openings 132 is sized, shaped and configured to receive a portion of the clasp 116 therein. Each jaw 114 includes an engaging portion 134 defined via the opening 132 and an exterior edge 150 of the jaw 114—i.e., an edge of the jaw 114 opposite the gripping features 146 of the jaw 114. As will be described in further detail below, the engaging portion 134 may be enclosed between the first and second arms 120, 122 in the engaging configuration. In one embodiment, the opening 132 extends through each of the jaws 114 midway between the first and second ends 142, 144 so that each opening 132 extends through opposing portions of the clip 102.

As discussed above, the clip 102 may be mounted to the insertion device 104 (e.g., an endoscope 106), as shown in FIGS. 1 and 2. The clip 102 may be mounted to the endoscope 106 via the adapter 110, which is sized, shaped and configured to be mounted over the distal end 108 of the endoscope 106. As will be understood by those of skill in the art, the endoscope 106 of this embodiment is configured to be inserted through a body lumen to a target area within the lumen and is sufficiently flexible to navigate through even tortuous paths of the body lumen.

The adapter 110 extends from a proximal end 152 to a distal end 154 and includes a channel 156 extending therethrough. A proximal portion 153 of the adapter 110 is configured to be mounted over the distal end 108 of the endoscope 106 while a distal portion 155 is configured to receive the clip 102 thereover in the insertion configuration. The proximal portion 153 of the adapter 110 may be mounted to the endoscope 106 via, for example, a friction fit, so that the channel 156 of the adapter 110 is substantially longitudinally aligned with a channel of the endoscope 106 and the proximal and distal ends of the adapter 110 are open so that items or suction applied to the distal end of the endoscope 106 (e.g., via a working channel of the endoscope 106) may pass through the adapter 110 to the target tissue. In addition, this permits tissue to be viewed through the channel 156 via an optical system in the distal end of the endoscope 106. In another embodiment, to enhance a visibility of the tissue and/or the clip 102, the adapter 110 may be formed of a transparent material.

In one embodiment, the proximal portion 153 of the adapter 110 may include a pair of holes 158 extending longitudinally through a wall 160 thereof. Each of the holes 158 is configured to slidably receive a corresponding one of the extending member 112 therein. As will be described in further detail below, the extending members 112 are received within the holes 158 so that the clasps 116 at the distal ends 118 of the extending members 112 extend distally of the holes 158 to engage the clip 102, which is mounted over the distal portion 155 of the adapter 110.

An outer diameter of the distal portion 155 of the adapter 110 is sized, shaped and configured to receive the clip 102 thereover, in the insertion configuration. In one exemplary embodiment, the distal portion 155 tapers toward the distal end 154 so that the bias of the clip 102 as it squeezes closed draws the clip 102 distally over the adapter 110 toward the initial deployed configuration either on its own as the extending members 112 are extended distally to free the clip 102 to move distally or assisting the distal movement of the clip 102 over the adapter as the extending members 112 push the clip 102 distally.

When the clip 102 is mounted over the distal portion 155 of the adapter 110 with each of the jaws 114 extending over opposing portions thereof, an exterior surface of the adapter 110 constrains the clip 102 in the insertion configuration, in which the jaws 114 of the clip 102 are separated from one another in the open configuration. The clip 102 remains mounted over the adapter 110 in the open insertion configuration so long as a sufficient proximally directed tension is applied to the extending members 112, while the clasps 116 engage the clip 102, so that the extending members 112 hold the clip 102 in place. If this tension is removed from the extending members 112, a natural bias of the clip 102 will draw the jaws 114 toward one another pushing the clip 102 distally over the tapered surface of the adapter 110 until the clip 102 slides distally off of the adapter 110 as the extending members 112 are moved distally with the clip 102.

As shown in FIG. 4, the distal portion 155 of the adapter 110 in this embodiment includes a plurality of longitudinally extending flat portions 162 distributed about the circumference of the exterior surface 148 of the adapter 110. Each of the flat portions 162 extends along at least a portion of a length of the distal portion 155 and covers a portion of a perimeter (e.g., circumference) of the distal portion 155. Flat portions 162 may be equally sized and separated from one another about the circumference of the distal portion 155. It will be understood by those of skill in the art that these flat portions 162 by remaining out of contact with the clip 102 reduce friction between the clip 102 and the distal portion 155 of the adapter 110 to facilitate movement of the clip 102 over the adapter 110.

The distal portion 155 of the adapter 110 of this embodiment also includes a plurality of projections 164 extending radially into the channel 156 of the adapter 110. In one embodiment, the plurality of projections 164 extend from curved portions 163 of the distal portion 155, which extend between adjacent flat portions 162. In one exemplary embodiment, a distal face 166 of each of these curved portions 163 is angled with respect to a longitudinal axis of the adapter 110 so that, when the clip 102 is drawn proximally from the initial deployed configuration and the jaws 114 abut the distal face 166, the angled of these projections 164 facilitate the re-opening of the jaws 114, against their natural bias, toward the open insertion configuration. That is, the jaws 114 slide proximally over the projections 164 to open the jaws 114 so that the clip 102 is released from the clipped tissue and can be slid proximally back into the adapter 110.

The insertion device 104 includes extending members 112 extending along a length of the endoscope 106 from proximal ends 168 accessible to the user via, for example, the user interface 138, to the distal ends 118. In an exemplary embodiment, the extending members 112 are formed as flexible strands, filaments or coils formed of, for example, a metal or polymer. In one embodiment, the insertion device 104 includes two extending members 112, each of the extending members 112 configured to be releasably coupled to a corresponding one of the jaws 114 via the clasp 116 at each of the distal ends 118.

Each extending member 112 in this embodiment extends through, for example, an outer shaft 170 extending along a length of the endoscope 106 from a proximal end 172 connected to a portion of the user interface 138 to a distal end 174 connected to the adapter 110. In particular, the distal ends 174 of the outer shafts 170 are connected to the proximal end 152 of the adapter 110 so that each outer shaft 170 is aligned with a corresponding one of the holes 158 of the adapter 110. Thus, each extending member 112 extends through a corresponding one of the outer shafts 170 and the holes 158 so that the extending member 112 is longitudinally movable therewithin, relative to the endoscope 106, to control movement of the clip 102 relative to the endoscope 106.

In one embodiment, each of the outer shafts 170 (and the corresponding one of the extending members 112) extends along opposing longitudinal sides of the exterior of the endoscope 106. Each of the outer shafts 170 in this embodiment is substantially longitudinally aligned with one of the holes 158 of the adapter 110 and one of the openings 132 of a corresponding one of the jaws 114 so that, when the clip 102 is mounted over the adapter 110, the clasps 116 at the distal ends 118 of the extending members 112 extend distally from the holes 158 of the adapter 110 to releasably engage the jaws 114 of the clip 102. It will be understood by those skilled in the art, however, that any arrangement of the outer shafts 170 may be employed so long as the extending members 112 are slidably housed therein and are delivered to desired locations on the clip 102 as described above.

As described above and as shown in FIG. 5, each clasp 116 includes the first arm 120 and the second arm 122, which are pivotally coupled to one another at the proximal ends 124, 126, respectively, thereof so that the first and second arms 120, 122 are movable between the engaging configuration and the disengaging configuration. For example, the clasp 116 of this embodiment includes a pin 123 pivotally connecting the first and second arms 120, 122 to one another so that the first and second arms 120, 122 rotate about the pin 123 to move between the engaging and disengaging configurations. According to an exemplary embodiment, the pin 123 also connects the first and second arms 120, 122 to a housing 117 of the clasp 116, which is fixed with respect to the distal end 118 of a corresponding one of the extending members 112. Thus, while the clasp 116 is movable between the engaging and disengaging configurations, the clasp 116 is not longitudinally movable relative to the extending members 112.

In one embodiment, the first arm 120 is hook-shaped, extending from the proximal end 124 along a substantially J-shaped curve to a hook tip at the distal end 128. In the engaging configuration, the hook tip at the distal end 128 of the first arm 120 contacts the distal end 130 of the second arm 122 so that the first and second arms 120, 122 together form an enclosure within which the engaging portion 134 of the corresponding jaw 114 is received. In particular, the hook tip at the distal end 128 is inserted into the opening 132 of the corresponding jaw 114 so that the engaging portion 134 is received within the curvature of the hook-shaped first arm 120. The distal end 130 of the second arm 122 contacts the hook tip distal end 128 of the first arm 120 to ensure that the engaging portion 134 of the jaw 114 is enclosed between the first and second arms 120, 122. When it is desired to release the clip 102 therefrom, however, each of the clasps 116 is moved toward the disengaging configuration in which the first and second arms 120, 122 are moved away from one another so that distal ends 128, 130 of the first and second arms 120, 122 are separated from one another so that the engaging portions 134 are released from the clasps 116 via the space between the first and second arms 120, 122.

Movement of the first and second arms 120, 122 is controlled via the control wire 136, which extends from a proximal end 192 connected to a portion of the user interface 138 to a distal end 194 connected to the proximal ends 124, 126 of the first and second arms 120, 122. The control wire 136 extends longitudinally through the extending members 112 so that the control wire 136 is longitudinally movable relative thereto. In an exemplary embodiment, the control wire 136 is connected to the first and second arms 120, 122 so that distal movement of the control wire 136 relative to the extending members 112 moves the clasp 116 from the engaging configuration to the disengaging configuration. According to one exemplary embodiment, the distal end 194 of the control wire 136 is connected to the proximal ends 124, 126 of the first and second arms 120, 122 via a four-bar mechanism. In this embodiment, portions of the first and second arms 120, 122 extending proximally of the pin 123 act as the first and second bars of the four-bar mechanism.

The clasp 116 further includes a third bar 176 and a fourth bar 178, proximal ends of which are pivotally coupled to the distal end 194 of the control wire 136 so that distal ends of the third and fourth bars 176, 178 are connected to a corresponding one of the first and second bars (e.g., proximal ends 124, 126 of the first and second arms 120, 122) via joints about which the bars may rotate relative to one another. Thus, longitudinal movement of the control wires 136 relative to the extending members 112 moves the first and second arms 120, 122 between the engaging and disengaging configurations. For example, when each control wire 136 is moved distally with respect to the corresponding extending member 112, distal ends of the third and fourth bar 176, 178 are moved away from one another, causing proximal ends of the first and second bars (e.g., portions of the first and second arms 120, 122 extending proximally of the pin 123) to also be moved away from one another so that the clasp 116 is moved from the engaging configuration toward the disengaging configuration.

Similarly, when each control wire 136 is moved proximally with respect to the corresponding extending member 112, the first and second arms 120, 122 move from the disengaging configuration toward the engaging configuration. While the exemplary embodiments show and describe thee control wires 136 connected to the first and second arms 120, 122 via a four bar mechanism, it will be understood by those of skill in the art that the control wires 136 may be connected to the arms 120, 122 of the clasps 116 via any of a variety of mechanisms so long as a longitudinal movement of the control wires 136 relative to the extending members 112 results in controlling movement of the clasps 116 between the engaging and disengaging configurations.

According to an exemplary embodiment, as shown in FIGS. 6-8, the user interface 138 includes a handle member 180, a spool 182 slidably mounted thereover, and a button 184 slidably coupled to the spool 182. The spool 182 is movable relative to the handle member 180 to move the clip 102 between the insertion configuration, the initial deployed configuration and the review configuration. The button 184 is movable relative to the spool 182 to move the clasps 116 between the engaging and disengaging configurations—e.g., from the review configuration toward the final deployed configuration.

The handle member 180 is connected to the proximal ends 172 of the outer shafts 170 while the spool 182 is connected to the proximal ends 168 of the extending members 112. In one embodiment, the proximal ends 172 of the outer shafts 170 are connected to the handle member 180 via an end cap 188 attached to a distal end 190 of the handle member 180. Proximal ends 172 may be fixed within the end cap 188 via any of a number of different mechanisms. For example, the proximal ends 172 may be crimp attached, welded or adhered to the end cap 188.

Thus, moving the spool 182 longitudinally over the handle member 180 corresponding moves the extending members 112 longitudinally through the outer shafts 170 relative to the endoscope 106. While the clip 102 is engaged via the clasps 116 at the distal ends 118 of the extending members 112, the spool 182 may be moved relative to the handle member 180 to move the clip 102 between the insertion configuration, the initial deployed configuration, and the review configuration.

The button 184 is connected to proximal ends 192 of the control wires 136 and may be slidably mounted relative to the spool 182 so that longitudinal movement of the button 184 relative to the spool 182 moves the first and second arms 120, 122 of the clasps 116 between the engaging and disengaging configurations. In one exemplary embodiment, the button 184 may be biased toward, for example, a proximal end of the spool 182 so that the clasps 116 are biased toward the engaging configuration. The button 184 may be biased toward the engaging configuration via, for example, a spring housed within the spool 182. Thus, a user of the system 100 is not required to hold the button 184 toward the engaging configuration and will only be required to slide the button 184 relative to the spool 182 when it is desired to move the clip 102 from the review configuration toward the final deployed configuration.

When it is desired to move the clip 102 toward the final deployed configuration, the user may slide the button 184 distally with respect to the spool 182 to correspondingly move the control wire 136 for each of the clasps 116 distally with respect to the extending members 112. The clasps 116 are then moved from the engaging configuration toward the disengaging configuration so that the engaging portions 134 of the clip 102 are released from between the first and second arms 120, 122 of the clasps 116, thereby deploying the clip 102 in the body.

According to another exemplary embodiment, the user interface 138 also includes a sliding lock 186 configured to limit a distal movement of the spool 182 relative to the handle member 180. In particular, the sliding lock 186 is slidable along a length of the handle member 180 to indicate a desired distance of the clip 102 from the adapter 110, when the clip 102 is in the review configuration. Once the position of the sliding lock 186 relative to the handle member 180 has been determined, to move the clip 102 from, for example, the initial deployed configuration to the review configuration, the user may slide the spool 182 distally relative to the handle member 180 until a portion of the spool 182 abuts the sliding lock 186.

The sliding lock 186 prevents the spool 182 from being moved any further distally so that, when it is desired to move the clip 102 from the review configuration toward the final deployed configuration, the sliding lock 186 holds the spool 182 in place so that the button 184 may be slid distally relative to the spool 182 to move the clasps 116 from the engaging configuration toward the disengaging configuration. The sliding lock 186 also provides the benefit of freeing up the user's hand. In particular, the user is not required to hold the spool 182 in the desired position relative to the handle member 180 so that the user is free to manipulate the system in other ways. For example, while the clip 102 is held in the review configuration via the sliding lock 186, the user may steer the distal end 108 of the endoscope 106 to view the clip 102 or the tissue from another angle.

According to an exemplary method for tissue closure utilizing the clipping system 100, the clip 102 may be inserted through a body lumen such as, for example, the GI tract, to a target area within the body lumen via the insertion device 104 which, in one embodiment, may include the endoscope 106. As described above, in the insertion configuration, the clip 102 is mounted to the distal end 108 of the endoscope 106 via the adapter 110, so that the jaws 114 are separated from one another toward the open configuration. The clip 102 is guided to the target area via the visualization system of the endoscope and positioned over a target tissue. A suction force and/or tissue graspers may be applied through a working channel of the endoscope 106 so that the target tissue may be drawn into the channel 156 of the adapter 110. Thus, when the clip 102 is moved toward the initial deployed configuration by releasing a tension along the extending members 112 (e.g., moving the extending members 112 distally relative to the endoscope 106), the clip 102 slides distally along the adapter 110 toward the biased closed configuration to grip the target tissue.

It will be understood by those of skill in the art that suctioning and/or gripping of the tissue in this initial deployed configuration may obstruct an imaging/optical lens of the endoscope 106 so that the user is unable to visualize and/or confirm whether a desired target tissue has been properly clipped. Thus, the clip 102 may be moved toward the review configuration by drawing the endoscope 106 proximally relative to the clip 102, while the clip 102 remains engaged with the clasps 116 and is clipped on the tissue. A distance between the adapter 110 and the clip 102 widens a field of view of the endoscope 106 so that the clip 102, and the tissue gripped thereby, may be viewed via the optical/visualization system of the endoscope 106.

If, upon visualization, the user determines that the clip 102 requires an adjustment and/or a repositioning relative to the target tissue, the extending members 112 may be translated proximally relative to the endoscope 106 until the clip 102 is moved proximally over the adapter 110, as described above, toward the open insertion configuration. In particular, the endoscope 106 may be moved distally relative to the extending members 112 while drawing the spool 182 proximally relative to the handle member 180 so that the clip 102 and the adapter 110 are drawn toward one another via the extending members 112. As the clip 102 is moved toward the open configuration, the tissue gripped thereby is released, permitting the clip 102 to be repositioned over the target tissue, as desired. The clip 102 may then once again moved toward the initial deployed configuration, and then again toward the review configuration. This process may be repeated, as necessary, until the user is able to visually confirm that the clip 102 has been clipped over the target tissue, as desired.

As discussed above, the clasps 116 remain engaged with the clip 102 during movement of the clip 102 relative to the endoscope 106 between the insertion, initial deployed and reviewed configurations. In particular, the engaging portions 134 of the clip 102 are enclosed between the first and second arms 120, 122 of the clasps 116. Once the user confirms that the target tissue has been clipped, as desired, the clip 102 may be moved from the review configuration toward the final deployed configuration, by moving the clasps 116 from the engaging configuration toward the disengaging configuration to release the clip 102 from the extending members 112.

As described above, according to an embodiment, the extending members 112 are maintained in position relative to the endoscope 106 so that the control wires 136 may be moved distally relative to the extending members 112 to move the first and second arms 120, 122 from the engaging configuration toward the disengaging configuration in which distal ends 128, 130 of the first and second arms 120, 122 are separated from one another to release the engaging portions 134 of the clip 102 therefrom. Thus, the insertion device 104, including the extending members 112, may be withdrawn proximally away from the clip 102 and out of the body, leaving the clip 102 clipped over the target tissue in the final deployed configuration.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-14. (canceled)

15. A clipping system for treating tissue, comprising:
an adapter including a proximal portion configured to he mounted over a distal end of an insertion device and a distal portion extending distally from the proximal portion;
a clip configured to be mounted over the adapter, the clip including first and second jaws connected to one another such that the first and second jaws are movable between an insertion configuration, in which the first and second jaws extend about opposing portions of the distal portion of the adapter and are separated from one another to receive tissue therebetween, and an initial deployed configuration, in which the clip is moved distally off of the adapter so that the first and second jaws are drawn toward one another to grip tissue therebetween, the first and second jaws being biased toward the initial deployed configuration; and
a first extending member configured to be releasably coupled to the clip and movably connected to the adapter, the first extending member including a clasp at a distal end thereof, the clasp including a first arm and a second arm pivotally coupled to one another so that distal ends thereof are movable toward and away from one another between an engaging configuration and a disengaging configuration, respectively, the clasp of the first extending member engaging an engaging portion of the first jaw to permit movement of the adapter relative to the clip while the first extending member remains coupled to the clip to place the system in a review configuration in which the clip is physically separated from the adapter to enhance visual observation of the clip, the first extending member being operable to retract the clip proximally over the adapter so that the clip is forced open as the clip is retracted over the adapter freeing the clip from tissue on which it had been clipped.

16. The system of claim 15, wherein the first jaw includes an opening extending therethrough, the engaging portion extending between the opening of the first jaw and an exterior edge of the first jaw.

17. The system of claim 16, wherein the first arm of the clasp is substantially hook-shaped, the first arm extending along a curve from a proximal end toward a distal hook tip, the first arm configured to be hooked through the opening of the first jaw so that the engaging portion of the first jaw is received within the curve of the first arm.

18. The system of claim 17, wherein proximal ends of the first and second arms of the clasp are pivotally coupled to one another so that the first and second arms pivot between the engaging configuration, in which the distal hook tip and a distal end of the second arm contact one another to enclose the engaging portion of the first jaw therewithin, and the disengaging configuration, in which the distal hook tip and the distal end of the second arm are separated from one another to release the engaging portion of the first jaw therefrom.

19. The system of claim 15, wherein the insertion device further includes a first control wire slidably received within the first extending member, a distal end of the first control wire connected to the first and second arms of the clasp at the distal end of the first extending member.

20. The system of claim 19, wherein the first control wire is connected to the clasp of the first extending member via a four-bar mechanism so that a longitudinal movement of the first control wire relative to the first extending member moves the clasp of the first extending member between the engaging and disengaging configurations.

21. The system of claim 15, wherein the proximal portion of the adapter includes a first hole extending longitudinally through a wall thereof to slidably receive the first extending member therein such that the clasp at the distal end of the first extending member extends distally of the first hole to engage the clip.

22. The system of claim 15, further comprising a second extending member configured to be releasably coupled to the clip and movably connected to the adapter, the second extending member including a clasp at a distal end thereof, the clasp of the second extending member including a first arm and a second arm pivotally coupled to one another so that distal ends thereof are movable toward and away from one another between an engaging configuration and a disengaging configuration, respectively, the clasp of the second extending member engaging an engaging portion of the second jaw to permit movement of the clip relative to the adapter between the insertion configuration, the initial deployed configuration and the review configuration.

23. The system of claim 22, wherein the insertion device further includes a second control wire slidably received within the second extending member, a distal end of the second control wire connected to the first and second arms of the clasp at the distal end of the second extending member.

24. A clipping system for treating tissue, comprising:
an endoscope extending longitudinally from a proximal end to a distal end;
an adapter including a proximal portion and a distal portion, the proximal portion configured to be mounted over the distal end of the endoscope so that a channel of the adapter is aligned with a channel of the endoscope;
a clip configured to be mounted over the distal portion of the adapter, the clip including first and second jaws connected to one another such that the first and second jaws are movable between an insertion configuration, in which the first and second jaws extend about opposing portions of the distal portion of the adapter and arc separated from one another to receive tissue therebetween, and an initial deployed configuration, in which the clip is moved distally off of the adapter so that the first and second jaws are drawn toward one another to grip tissue therebetween, the first and second jaws being biased toward the initial deployed configuration, each of the first and second jaws including an opening extending therethrough to define an engaging portion; and
first and second extending members configured to be releasably coupled engaging portions of the first and second jaws of the clip and movably connected to the adapter, each of the first and second extending member including a clasp at a distal end thereof, each clasp including a first arm and a second arm pivotally coupled to one another so that distal ends thereof are movable toward and away from one another between an engaging configuration and a disengaging configuration, respectively, so that when the first and second extending members engage the clip in the engaging configuration, longitudinal movement of the first and second extending members relative to the endoscope moves the clip between the insertion configuration, the initial deployed configuration and a review configuration in which the clip is physically spaced from the adapter to enhance visual observation of the clip.

25. The system of claim 24, wherein the first arm of each clasp is substantially hook-shaped, the first arm extending along a curve from a proximal end toward a distal hook tip, the first arm configured to be hooked through a corresponding opening of one of the first and second jaws so that the engaging portion defined via the opening and an exterior edge of the corresponding one of the first and second jaws is received within the curve of the first arm.

26. The system of claim 25, wherein proximal ends of the first and second arms of each clasp are pivotally coupled to one another so that the first and second arms pivot between the engaging configuration, in which the distal hook tip and a distal end of the second arm contact one another to enclose the engaging portion of the corresponding one of the first and second jaws therewithin, and the disengaging configuration, in which the distal hook tip and the distal end of the second arm are separated from one another to release the engaging portion of the corresponding one of the first and second jaws therefrom.

27. The system of claim 24, wherein the endoscope further includes first and second control wires, the first and second control wires slidably received within the first and second extending members, respectively, so that distal ends of each of the first and second control wires are connected to the first and second arms of the clasp of each of the first and second extending members.

28. The system of claim 27, further comprising a user interface including a handle member, a spool slidably mounted over the handle member, and a button slidably coupled to the spool, the spool connected to proximal ends of the first and second extending members so that sliding a spool longitudinally relative to the handle member correspondingly moves the first and second extending members relative to the endoscope, the button connected to proximal ends of the first and second control wires so that siding the button longitudinally relative to the spool correspondingly moves the first and second control wires relative to the first and second extending members.

29. A method for treating tissue, comprising:
inserting a clip to a target area in a body lumen via an endoscope, the clip mounted over a distal end of the endoscope, via an adapter, in an open insertion configuration in which first and second jaws of the clip are separated from one another;
drawing tissue into a channel of the adapter and between the jaws of the clip;
moving the clip from the open insertion configuration toward an initial deployed configuration by releasing a tension along extending members coupled to the clip so that the jaws revert to a biased closed configuration, in which the jaws extend toward one another to grip the tissue received therebetween, the extending members releasably coupled to the clip via clasps at distal end of the extending members, each of the clasps including first and second arms pivotally coupled to one another, distal ends of the first and second arms contacting one another to enclose engaging portions of the clip therebetween in an engaging configuration; and
moving the endoscope relative to the clip, while the extending members remain coupled to the clip, to a review configuration in which visualization of the clip via the endoscope is enhanced.

30. The method of claim 29, wherein, when it is determined that the clip requires repositioning, moving the extending members proximally relative to the endoscope until the clip is drawn proximally over the adapter toward the open insertion configuration and repositioning the clip over the target tissue.

31. The method of claim 29, further comprising moving the clip from the review configuration toward a final deployed configuration by moving the clasps toward a disengaging configuration, in which distal ends of the clasps are moved away from one another toward a disengaging configuration to release the engaging portions of the clip from therebetween.

32. The method of claim 31, wherein the clasps are moved between the engaging configuration and the disengaging configuration via control wires connected to proximal ends of the first and second arms of the clasps, the control wires slidably received within the extending members so that the control wires are longitudinally movable relative to the extending members.

33. The method of claim 29, wherein the first arm of each clasp is substantially hook-shaped so that the first arm extends along a curve from a proximal end toward a distal hook tip, the first arm hooked through a corresponding opening of one of the first and second jaws so that the engaging portions defined between the opening and an exterior edge of the corresponding one of the first and second jaws is received within the curve of the first arm in the engaging configuration.

34. The method of claim 29, wherein the endoscope is moved relative to the clip to the review configuration by advancing the extending members distally relative to the endoscope.
